(19) United States
(12) Patent Application Publication
(10) Pub. No.: US 2026/0193718 A1
(43) Pub. Date: Jul. 9, 2026

(54) HIGH INPUT, HIGH SENSITIVITY ASSAYS

(71) Applicant: SAGA Dx, Inc., Cambridge, MA (US)

(72) Inventors: Samuel Woodhouse, Lund (SE); Anthony Miles George, Lund (SE); Miguel Alcaide, Lund (SE); Yilun Chen, Lund (SE); Lucia Oton, Lund (SE)

(21) Appl. No.: 19/442,688

(22) Filed: Jan. 7, 2026

Related U.S. Application Data
(60) Provisional application No. 63/743,147, filed on Jan. 8, 2025.

(51) Int. Cl. C12Q 1/6886; C12Q 1/6806; C12Q 1/6809; C12Q 1/6827; C12Q 1/686; C12Q 1/6869

(52) U.S. Cl. CPC C12Q 1/6886; C12Q 1/6806; C12Q 1/6809; C12Q 1/6827; C12Q 1/686; C12Q 1/6869

(57) ABSTRACT

Assays for minimal residual disease (MRD) that use at least 100 ng of cfDNA extracted from bodily fluid (e.g., 1.5 μg) as input and use digital PCR in which a sample is divided into compartments provided with PCR primers that are designed to specifically amplify circulating tumor DNA (ctDNA). PCR is performed with tumor-specific detectably-labeled probes such that detecting signal from the compartments provides evidence of ctDNA in the bodily fluid and thus evidence of MRD in the patient.

### TECHNICAL FIELD

The disclosure relates to identifying and detecting variants as biomarkers of diseases.

### BACKGROUND

Health care providers have numerous approaches to detecting and treating cancer. Cancer may be detected using physical examination or imaging to detect a lump or tumor. In some cases, cells are examined under a microscope by a cytopathologist for features associated with cancer. Once cancer is detected, potential treatments include surgery to remove a tumor, radiation therapy, and immunotherapy. When treating cancer, it is hoped that the treatment will completely remove all tumor cells, allowing the patient to live a long life, free of cancer. Unfortunately, sometimes a small number of cancer cells remain in the body after treatment, a phenomenon known as minimal residual disease (MRD). Patients would have the best outcomes if health care providers had highly sensitive tests for MRD.

It is understood that tumor cells release small mount of tumor DNA to circulate in the bloodstream among other cell-free DNA (cfDNA). That circulating tumor DNA (ctDNA) is a promising biomarker for detection of MRD. Cell-free DNA (cfDNA), including ctDNA, is released from cells into bodily fluids such as circulating blood. A significant challenge in the use of ctDNA for liquid biopsy (e.g., blood or plasma samples) is the low number of target ctDNA molecules in blood compared to the (relatively) high amount of cfDNA other than ctDNA. In fact, some events and conditions promote the release of an abundant amount of non-cancer cfDNA, a phenomenon sometimes called cfDNA leakage. For example, the surgical removal of a tumor contributes significantly to cfDNA leakage. It is thought that in the week to ten days after surgical tumor resection, such an abundance of non-tumor DNA is released that molecular assays for ctDNA as a test for MRD may be prone to false negatives, or inadequate sensitivity, because the cfDNA load associated with cfDNA leakage masks any positive results from the ctDNA-based MRD assay.

### SUMMARY

The invention provides molecular assays for minimal residual disease (MRD) that use, as input, a quantity of cell-free DNA (cfDNA) significantly higher than the 30 to 70 ng that have conventionally been used in such an assay. Compared to conventional MRD assays that use no more than 70 ng of cfDNA, methods of the invention use at least 100 ng of cfDNA extracted from bodily fluid and preferably use on the order of 1 μg even, for example, 1.5 μg or more as input into an MRD assay. Assays for MRD of the invention may make use of digital PCR in which a sample is divided into compartments (either solid or aqueous) at a dilution such that, on average, each compartment receives zero or one molecule of cfDNA. The compartments are also provided with PCR primers that are designed to specifically amplify circulating tumor DNA (ctDNA) from a tumor for which the patient has been treated. The compartments are subject to an amplification reaction (e.g., PCR) in the presence of labeled probes that give a detectable signal when the ctDNA is amplified. Detecting the detectable signal from the compartments provides evidence of the ctDNA in the bodily fluid sample and thus evidence of MRD in the patient.

The described MRD assays have high sensitivity, and avoid false negatives, through the use of high quantities of cfDNA, or amplification products made therefrom, as input into the assays. By loading the assays with quantities of input DNA beyond what has conventionally been expected to work, the likelihood of detecting very rare ctDNA molecules is increased and false negatives are avoided. Methods of the invention may include any of a number of technical features to match the input DNA to the purpose of the MRD assay. For example, a sample comprising cfDNA extracted from bodily fluid may be subject to a tumor DNA-specific pre-amplification to increate a proportion of tumor sequences relative to all nucleic acid sequences within the sample. The pre-amplification produces a set of input DNA that includes a mixture of the original cfDNA and copies made from ctDNA and has at least about 100 ng, even as much as 1 microgram of that DNA, that is loaded into the MRD assay.

Where the MRD assay uses digital PCR (dPCR), one feature by which the input DNA quantity may be matched to the purpose of the assay involves loading only a portion (e.g., half) of the input sample into the compartments. If the dPCR gives a result indicating saturation, e.g., a majority or more of compartments giving a positive signal, then a second portion of the sample may be diluted (by a known dilution factor) and divided into a second set of the compartments for a second dPCR readout. Using the known dilution factor (and any information from a result of any pre-amplification reaction), a calculation based on a number of compartments giving a positive result in the second dPCR gives a quantity of ctDNA in the bodily fluid sample. By those features and means, methods of the invention provide highly sensitive molecular assays for MRD.

Because those MRD assays are tailored or adjusted to improve detection of very small quantities of ctDNA among high quantities of cfDNA, MRD assays of the disclosure are well suited to detect MRD under conditions of cfDNA leakage. Thus, in the hours or days after treatment to remove a tumor, an MRD assay may be performed according to methods described herein to detect the continued presence of viable cancer cells, e.g., MRD, despite the recent treatment to remove the tumor. In fact, other biological and physiological conditions are known or suspected to promote cfDNA leakage including, for example, acute tissue injury, age-related degeneration, arthritis, pregnancy, infection, and inflammatory conditions. When a person has been treated to remove a tumor or is otherwise suspected to be experiencing a condition associated with cfDNA leakage, an MRD assay of the invention may be performed to detect and evaluate the success of the cancer treatment. Finding evidence of the presence of ctDNA can guide and inform health care providers in selecting further treatment options to ensure a healthy life for the patient.

In certain aspects, the invention provides methods for detecting a tumor. Methods include dividing at least a portion of a sample into partitions. The sample comprises at least 100 ng of cell-free DNA from a subject. The methods include performing an amplification reaction in the partitions using reagents that specifically amplify tumor DNA and that provide a detectable signal when the tumor DNA is amplified. The presence of MRD in the subject is reported when the detectable signal is detected.

Methods may include conducting a pre-amplification reaction in the portion of the sample. Preferably, the pre-amplification reaction uses primers that specifically amplify the tumor DNA. After the pre-amplification, the portion that gets divided into partitions preferably includes at least 100 ng of DNA, which may include either or both of the cell-free DNA and/or product of the pre-amplification. In some embodiments, after the pre-amplification, the portion (that is divided into partitions) includes at least 1 μg of DNA. Preferably, the sample includes between and 1 and 2 μg of the cell-free DNA and/or copies thereof.

In certain embodiments, the methods are suitable for conditions associated with cfDNA leakage. For example, the sample may have been obtained within about 10 days or less after a treatment to remove a tumor from the subject.

In some embodiments, methods include a dilution step if (i.e., when) digital PCR exhibits saturation. For example, the portion may include about half or less of the at least 100 ng of the cell-free DNA and the method may include, when at least a majority of the partitions give the detectable signal (i.e., “saturation”), taking a second portion of the cell-free DNA, diluting the second portion, dividing the second portion in a second set of partitions, and repeating the amplification reaction.

For certain minimal residual disease (MRD) embodiments, the subject has undergone treatment to remove a tumor and the method further includes—prior to the dividing step: analyzing tumor DNA from a tumor sample from the subject to detect tumor-specific sequences; designing primers specific for the tumor-specific sequences; and performing the subjecting the partitions to the conditions for the amplification reaction with the primers specific for the tumor-specific sequences. The analyzing step may include: sequencing tumor DNA from the tumor sample to obtain tumor sequence reads; and comparing the tumor sequence reads to reference to identify one or more variants in the tumor sequence reads, compared to the reference. Methods may include filtering the identified one or more variants against data describing benign somatic variants to retain only tumor-specific variants as the tumor-specific sequences. The data describing the benign somatic variants may comprise matched-normal sequence reads from non-tumor tissue from the subject and/or a database of somatic variants. Preferably the tumor-specific sequences comprise structural variants (SVs) specific to the tumor, or breakpoints of the SVs.

In methods of the invention, the portion of the sample that is divided into partitions may include more than 1 μg of the cell-free DNA (e.g., even about 1.5 microgram). Dividing the portion of the sample into the partitions may include forming droplets or dividing the at least the portion into wells of a plate. The method may include analyzing the partitions with a digital PCR instrument.

In certain embodiments, the partitions, the conditions for the amplification reaction, and the reagents that specifically amplify the tumor DNA constitute a digital PCR assay for minimal residual disease (MRD). The digital PCR assay for the MRD may be performed using an input amount of DNA that is loaded into the partitions, in which the input amount of the DNA is between about 1 and 2 μg, optionally between about 1 and 1.5 μg. The DNA may include the cell-free DNA from a subject and/or product from a pre-amplification reaction that specifically pre-amplifies the tumor DNA from among the cell-free DNA.

### DETAILED DESCRIPTION

MRD assays using e.g., digital PCR (dPCR) and/or next-generation sequencing (NGS) based approaches have conventionally been understood to have maximum input between about 30 ng and 70ng of nucleic acid. Upper limits on DNA quantity have been related to concepts such as dPCR saturation or coverage depth for NGS. For example, when increasing input DNA quantity, one typically must increase the sequencing depth to account for the increased number of molecules that need to be “sampled”. Increasing input is also hampered by the underlying error/specificity of NGS assays. Therefore, people have not used high quantities such as 1.5 μg of DNA as input into a single assay. Methods of the invention use up to 1.5 μg input into a single MRD assay.

Having a variable input of between 5 ng to 1.5 μg (e.g., an DNA input quantity from about 100 ng to about 1.5 μg) into an MRD assay has several advantages. Using such an input quantity maximizes sensitivity of the assay. As more cfDNA input is loaded into assay, the chance of having a molecule of ctDNA in the total cfDNA is increased (which improves the chance of a true positive). Due to the high input quantity, the assay is robust to cfDNA leakage. Under cfDNA leakage, with a conventional (e.g., sub 70 ng) cap on input DNA, increased cfDNA can drown out a positive signal (leading to a false negative call). Those issues are particularly applicable shortly, e.g., days, after surgery, when large amounts of cfDNA flood into the blood system masking the presence of ctDNA molecules.

Methods of the invention work well with high quantities of input DNA and may include additional features or steps to promote high sensitivity when using high quantities of input DNA. For example, methods herein may be designed and implemented so that the assay readout does not read and detect both mutant (tumor) and wild-type (homologous sequences from “matched normal” cfDNA). This may involve identifying variants in tumor DNA and filtering those variants against information describing known, benign somatic variants to identify only truly tumor-specific variants, allowing one to design reagents, e.g., primers, for an MRD assay that only detects tumor-specific sequences. Methods herein may be designed and implemented with approaches that avoid problems with “saturation” in digital PCR assays. In digital PCR (dPCR), it is possible that all compartment or partitions are positive, a phenomenon known as saturation. Here, saturation can be avoided by using a portion, e.g., half, of the starting sample as input into the dPCR run. If that dPCR run is saturated (all partitions give a signal), a second portion of the initial sample may be diluted by a known dilution factor and a second dPCR run may be performed. From the second dPCR run, a count of positive partitions may be divided by the dilution factor to give a quantitative estimate of target molecules in the starting sample.

Methods of the invention may include further or additional key steps. As discussed in greater detail herein, a bodily fluid sample, such as blood or plasma, is obtained, and cfDNA is extracted from the sample. Preferably up to 1.5. μg of cfDNA (e.g., a quantity between 100 ng and 1.5. μg) is extracted. By having analyzed tumor DNA from a known tumor sample, methods use primers that are designed to detect and amplify only tumor-specific variants, preferably structural variants (SVs) that are specific to the tumor. From the bodily fluid sample, a pre-enrichment may be performed, e.g., to specifically pre-amplify those tumor-specific variants, e.g., SVs. To address issues with saturation, methods may include, after enrichment, using 50% (or less than) of the material. If dPCR saturation occurs, a second portion of the extracted cfDNA is diluted, e.g., 1:10 or 1:50. The dPCR is conducted again, accounting for the dilution in any quantification step. Using features and techniques described herein, methods of detection exhibit a linear relationship between input and detection. For example, loading 300 ng instead of 150 ng provides a 2× increased sensitivity.

Methods of the disclosure are provided for performing an MRD assay where the ctDNA (or ctRNA) input into the detection assay is >100ng and quantification and detection occur to determine the level and ctDNA positivity of a sample. When tracking a patient using an MRD assay, there may be variable input quantities depending on the blood sample and time point. Ideally, all molecules are amplified, and then mutant molecules are detected in the sample. Noting that high cfDNA load after surgery masks a positive signal (i.e. large amounts of non-tumor DNA diluting out the ctDNA), methods herein are well-suited to performing a MRD assay<10 days after surgery, even wherein an entire sample from surgery is analyzed.

By the described techniques, the invention provides methods that are useful to detect tumor nucleic acid in a bodily fluid sample, using at least 100 ng of cfDNA extracted from the bodily fluid sample (or pre-amplified from such cfDNA) as input to the assay. The detection is specific for tumor nucleic acid by virtue of the use of primers designed by analyzing sequence data from tumor DNA obtained from a known tumor sample that has been obtained prior to tumor removal. The initial sequence data analysis, which may involve next-generation sequencing (NGS) of a tumor sample such as from a biopsy or a formalin-fixed, paraffin embedded (FFPE) tumor slice, and may proceed by low-pass, whole genome sequencing (LP-WGS), and may be performed at one point in time to detect a plurality of tumor-specific variants.

In various embodiments described herein, the sequence data may be obtained by sequencing DNA from an FFPE slice of the tumor; a library preparation protocol tailored to FFPE-sourced nucleic acid may be used; the tumor nucleic acid may be sequenced by LP-WGS; a computer system may be used to detect and rank tumor SVs and select a marker variant and to design primers specific for the marker variant; the primer pair may be used in a detection assay for a subject that has undergone treatment to eradicate the tumor; the sample may be a blood draw, or “liquid biopsy”, the detection assay may involve digital PCR with the sample in partitions using an amplification reaction and fluorescent probes; detecting fluorescence from the droplets may indicate the presence of the tumor nucleic acid in the sample; and/or the assay may be performed to detect minimal residual disease after the treatment.

### FFPE DNA Extraction

Methods of the disclosure may include obtaining nucleic acid from a formalin-fixed, paraffin embedded slice of a tumor so that the tumor nucleic acid may be sequenced. Tissue obtained by biopsy or surgery for pathological examination may be fixed in a fixative, such as formalin and embedded in paraffin, yielding formalin fixed, paraffin embedded (FFPE) blocks. Small (e.g., a few micrometer-thick) sections may be sliced from the blocks and stained on slides for microscopic analysis. Such slides are typically retained as a pathology archive.

Methods herein may use protocols for extracting DNA from FFPE samples and preparing high-quality sequencing libraries from the FFPE-extracted DNA. To extract nucleic acid, the sample is loaded into a tube such as microcentrifuge tube. A tissue lysis buffer and proteinase K (PK) solution mix may be added to the tube. Steps of protocols herein may be performed using reagents and material sold under the product name truXTRAC FFPE total NA (tNA) Ultra Kit by Covaris. The FFPE sample may be immersed in the tissue lysis buffer/PK solution mix and sonicated in a ultrasonication instrument according to manufacturer instructions for paraffin emulsification. The steps may be performed in laboratory test tubes, wells of a plate, microcentrifuge tubes, or tubes in a multi-tube strip.

After the tube is collect, it is centrifuged, e.g., spun at 5 k g for about 15 minutes, to form a pellet that includes DNA, preferably at least 100 ng, optionally even up to 1.5 μg. The described protocols provide high quality DNA, suitable or sequencing, with high yield (e.g., between 1 and 2 μg) from FFPE tissue samples. Preferably, the pellet is rehydrated with a suitable buffer such as buffer BE from Covaris and more preferably a tissue lysis buffer/PK solution mix is used. The tube may be sonicated to resuspend material of the pellet, and optionally treated with RNase. A DNA purification column may be placed into a collection tube. The sample is transferred into the column and the tube spun. Following DNA purification protocol instructions, the column is washed with buffer(s) such as BW Buffer and B5 Buffer (Covaris). Finally, the column is eluted with an elution buffer, eluting the DNA from the column. The collected (eluted) DNA preferably includes at least 100 ng, optionally about 1.5 μg, and may be analyzed or stored long-term. Methods of the disclosure produce high quality and high yield sequencing libraries from FFPE-extracted DNA.

### Library Preparation

Having extracted DNA from a sample, methods may include library preparation, which generally includes fragmentation, adaptor ligation, and amplification. When the source is a tumor biopsy, nucleic acids in very small quantities, or preserved (e.g., FFPE) sample, extracted DNA may be fragmented via a fragmentation step that may be more gentle and less damaging than conventional protocols. In some embodiments, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. In some embodiments, it may be preferable to produce fragments that have an average size with a peak approximately within the range of about 500, preferably at least about 600 or 700, and most preferably at least about 800 base pairs (bp) to 1,000 bp. A cocktail of restriction enzymes may be composed that will, on average, cut genomic DNA on about 800 to 1,000 base intervals. Preferred embodiments use a sonicator or adaptive acoustic focusing (AFA) instrument (Covaris). Embodiments may use a Qubit instrument to evaluate quantity and/or a TAPESTATION automatic electrophoresis instrument to evaluate fragment length, using manufacturer's literature for guidelines for the sonication instrument. One approach is to shear a very small sample to the desired optical density to establish the instrument settings to be used for the bulk of the sample. The resultant shearing protocol produces 800 to 1000 base fragments.

The fragments may be repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA repair enzymes and Structure-specific endonucleases are enzymes that cleave DNA at a specific DNA lesion or structure. Those enzymes can be used for repair of DNA from sample degradation due to oxidative damage, UV radiation, ionizing radiation, mechanical shearing, formalin fixation (post extraction) or long-term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

In some embodiments, the end-repaired fragments are purified using magnetic beads and a magnetic separation device. A bead to DNA fragment ratio of about 0.7× may be used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. One feature of embodiments of the disclosure may be a minimal or low-bead ratio, which, in combination with the fragment length and subsequent steps, provides high quality, high-yield sequencing libraries from FFPE samples. Enzymes or other reagents may be washed away, and DNA may be eluted into a ligation mix.

Methods may include ligating adaptors to the fragments to form adaptor-ligated fragments. Any suitable approach may be used. Some embodiments include dA tailing at the 3′ ends of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). The adaptor ligated fragments may be subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp.

The selected adaptor-ligated fragments may be amplified to obtain amplicons. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage. The result is a plurality of clonal amplicons copied from nucleic acid in a tumor sample. The amplicons may have sequencing adaptors or any suitable primer binding sites at either or both ends. At this stage, a library preparation is complete.

The described extraction and library preparation protocols may be optimized, compared to commercially available kits and protocols, to compensate for damage that is characteristic of FFPE samples and their extraction. For example, after emulsification of the paraffin, DNA may be subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of the steps—limited fragmentation, gentle bead clean-up, and purification after adaptor ligation with size-selection step—may contribute importantly to the preparation of high-quality sequencing libraries from FFPE samples.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline SVs. Such an analysis may provide an identification of tumor-specific somatic SVs that are present in a patient's tumor DNA. That information is then used to design reagents to assay future samples from the patient for those same tumor-specific somatic SVs. For example, an informatics pipeline may be used to design amplification primers and fluorescent probes for the detection of such variants by a digital PCR assay. Some embodiments identify tumor-specific SVs present in a patient's tumor DNA and then use an informatics pipeline to design primers and fluorescent probes useful for detecting by digital PCR those SVs in cell-free tumor DNA in blood or plasma, e.g., from a liquid biopsy.

### Sequencing

Nucleic acid obtained according to methods of the disclosure is preferably sequenced to obtain sequence data. For example, methods may include sequencing DNA from a tumor sample from the subject to obtain sequence reads.

Sequencing may be by any method known in the art. Suitable DNA sequencing techniques may include the dideoxy chain-termination sequencing technique known in the art as Sanger sequencing, which uses labeled terminators and gel separation in a slab or capillary. Sequencing may include the sequencing by synthesis using reversibly terminated nucleotides and the detection of pyrophosphate in the technique known as pyrosequencing commercialized by ROCHE 454. Sequencing may proceed by techniques that include allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes. Sequencing may be performed using one of the single molecule, long read sequencing platforms commercialized by HELICOS, PACIFIC BIOSCIENCES, or OXFORD NANOPORE.

Sequencing techniques and instruments that may be used include, for example, those offered by ILLUMINA, INC. or ULTIMA GENOMICS. Illumina sequencing is based on the amplification of a sequencing library described above on a solid surface of a flow cell using fold-back PCR and anchored primers. Amplicons of adaptor-ligated fragments that constitute the sequencing library are annealed to oligos attached to the surface of flow cell channels that are extended by which the amplicons are bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing generates sequence data and for short-read, ensemble sequencing platforms such as the ILLUMINA platform, the sequence data comprises a large number of short sequencing reads typically accessible from the ILLUMINA system in a computer file format known as FASTQ.

The sequencing instrument and technique relates to the biochemistry of base determination and also implicates read length and read number, with consequences for read assembly. For example, the output from Sanger sequencing on a glass-capillary instrument provided by ABI is typically a small number of medium length (several hundred bases) chromatograms that are provisionally “called” (interpreted) as bases by software and presented visually for human verification. Long read sequencing (e.g., PACIFIC BIOSCIENCES, OXFORD NANOPORE) is meant to provide single or low numbers of much longer (>1,000) base reads. Short read sequencing (e.g., ILLUMINA) provides a large number (e.g., millions) of short reads (e.g., 50 or fewer bases) that are typically mapped to a reference and/or assembled de novo to show the original sequence. Illumina is accepted as an industry standard example of a next-generation sequencing (NGS) platform. Whatever instrument or technique is used, methods may include one or any combination of suitable “coverage” strategies, which involve determinations of what targets to sequence and at what coverage.

Coverage strategies may include, for example, transcriptome sequencing in which all RNA transcripts are sequenced redundantly, re-sequencing in which a presumptively very similar genome is known and only highly variable targets are sequenced, whole exome sequencing in which all expressed genes or exons are sequenced, or other coverage strategies. Even with a particular coverage strategy, one may opt for a certain depth of coverage. For example, for some applications, when NGS is used, 30× coverage is considered a standard coverage in which substantially all bases are sequenced redundantly such that each base, on average, appears in about 30 unique sequence reads. Certain preferred embodiments of the invention use low-pass whole genome sequencing (as used herein, “whole genome sequencing” means that a substantial portion such as at least 70% or 90% of a genome or at least the equivalent of at least one chromosome is sequenced). Low-pass whole genome sequencing (LP-WGS) is a technique in which each base is sequenced a few times (known as low-depth coverage) e.g., with a depth of coverage below about 15, even 5, and as low as 0.1-1 times. By reducing the depth of coverage, the cost of sequencing the whole genome is reduced while maintaining genome-scale coverage. LP-WGS is described in Christodoulou, 2023, Combined low-pass whole genome and targeted sequencing in liquid biopsies for pediatric solid tumors, NPJ Precision Onc 7:21 and Zheng, 2022, Experience of low-pass whole genome sequencing-based copy number variant analysis, Diagnostics (Basel) 12 (5): 1098, both incorporated by reference.

Whatever technique and coverage are employed, methods include sequencing nucleic acid from a tumor. In certain preferred embodiments, LP-WGS is used to sequence substantially at least about 60 or 70 or 80 or 90% of a tumor genome at a coverage of about 15× or lower. The sequencing provides sequence data of the tumor nucleic acids. The sequence data may be analyzed to create a personalized tumor mutation profile, which includes any potential tumor variants and/or mutations. Low-pass sequencing is especially well suited for methods described herein where an objective is to detect SVs because, compared to other genotyping purposes, SVs are readily discoverable by LP-WGS as described herein.

A variety of different variants and mutations may be tracked using the tumor mutation profile. Typically, these variants are structural variants. Structural variants (SVs) are genomic abnormalities that may amplify, delete, or rearrange genomic regions of a tumor. It is possible and, in fact, common for more than one SV to occur in the same tumor. As used herein, an SV generally refers to a rearrangement, duplication, or deletion of a segment of length of at least about 1,000 bases. Methods of the disclosure may also be used to detect tumor-specific polymorphisms and/or small indels.

### Detection of Tumor-specific Variants

The disclosure includes methods for analyzing sequence reads, as may be obtained from nucleic acid from tumors, to identify structural variants (SVs), and optionally filter out any putative structural variants that are not somatic (e.g., germline SVs or artifacts from sample processing or sequencing) to identify SVs that are specific to the tumor, i.e., tumor variants. Methods may include comparing tumor sequence to a reference by one or more algorithms, identifying structural variants in the tumor nucleic acid, and designing primers to specifically amplify those tumor variants. Sequence reads from tumor nucleic acid may first be cleaned up, mapped to a reference, and or subject to computational workflows to detect SVs.

Reads can be cleaned using known software methods such as fastp as described in Chen, et al., 2018, fastp: an ultra-fast all-in-one FASTQ preprocessor, Bioinformatics, 34(17): i884-i890, incorporated by reference. Cleaning may include trimming adapter sequences, removing low quality bases at the ends of reads and artifacts such as polyG tails. In some FFPE embodiments cleaning may include removing reads shorter than 30 bp instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated by reference. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, each incorporated by reference. Sequence assembly or mapping may employ assembly steps, alignment steps, or both. Assembly can be implemented, for example, by the program ‘The Short Sequence Assembly by k-mer search and 3’ read Extension ‘(SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9): 1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

A workflow for SV detection from sequence reads and for primer design may be automated using tools such as Snakemake or Nextflow and custom programming using R or Python, for example, to link input/output across the various workflow steps. Some embodiments employ a computational pipeline that uses two or more different algorithms, each intended for finding SVs, to call putative SVs and merge the results. The computational pipeline may be used for mapping reads to a reference by a first algorithm (in a first mapping) and also by a second algorithm to identify SVs by each algorithm and then selecting the better result or merging the results of the multiple mapping steps to describe the structural variants. One of the algorithms may be a graph-based algorithm. In preferred embodiments, the first algorithm adds the reads to a genomic graph and finds a path through the graph best supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27(12): 2050-2060 and Cameron, 2021, GRIDSS 2:
comprehensive characterization of somatic structural variation using single breakend variants structural variant phasing, Genome Biol 22(1): 202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read pairs to a reference and searches for genomic regions in the reference where a significant number of read pairs align to the reference in positions inconsistent with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9): 677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7(8): 1034-1047, incorporated herein by reference in its entirety. SplitSeq can be used to reconstruct the exact fusion sequence based on split reads and read pairs with one unmapped mate. Discordant reads can be re-aligned to reduce false positive SV calls. After merging of the SV calling paths using the first and second algorithms, the putative SVs can be annotated with genes that overlap SV breakpoints,

Methods may include filtering SVs that were identified by the mapping workflows to remove germline SVs and/or sample handling artefacts, thereby providing a set of somatic SVs, or tumor variants, present in the tumor DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, for an MRD assay of the invention to achieve excellent sensitivity and specificity, all SVs found by sequencing are preferably filtered to remove benign germline variants from the putative set, leaving a set of tumor-specific somatic SVs. Filtering may include comparing to a database of known SVs to remove from consideration those that are documented to be benign. Such a database may include the Genome Aggregation Database (gnomAD) described in Chen, 2023, A genomic mutational constraint map using variation in 76,156 human genomes, Nature 625:92-100, incorporated by reference; Genome in a Bottle SVs described in Chapman, et al., 2020, A crowdsourced set of curated structural variants for the human genome, PLOS Comp Bio, 16(6): e1007933, incorporated by reference; or the database of human structural variation known as dbVar described in Lappalainen, 2013, DbVar and DGVa: public archives for genomic structural variation, Nucleic Acids Res 41(Databse Issue):D936-41, incorporated by reference.

The described workflows provide for mapping the sequence reads to a reference and identifying read mappings that indicate a structural variant in the tumor nucleic acid, relative to the non-tumor nucleic acid of the subject. That structural variant is tumor specific. It is a variant specific to the tumor, herein referred to as a tumor variant. Using methods of the disclosure, the tumor variant is found by sequencing tumor nucleic acid and analyzing the sequence data. A feature of the disclosure is that such a tumor variant may be confirmed by orthogonal testing. Thus, the invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### Selecting a Set of Marker Variants

Methods of the disclosure include a step of selecting from any and all detected tumor SVs a subset of those that constitute a set of marker variants to be used in a digital PCR MRD assay. Criteria for selecting the marker variants may include level of duplication; driver mutations; passenger mutations; truncal mutation; conservation across tumor clonality; suitability for PCR; other criteria; and any combination of the foregoing. The invention includes methods for ranking structural variants (SVs) and/or otherwise detecting and assigning relative ranks, in terms of selection for an MRD assay. Systematically ranking SVs provides an approach for the automatic selection of which SVs to interrogate in a diagnostic assay, such as a digital PCR assay for MRD from circulating-tumor DNA in blood or plasma. Methods include analyzing sequence data from tumor nucleic acid from a tumor of a subject to identify the presence and copy numbers of a plurality of tumor-specific structural variants (SVs) in the tumor nucleic acid compared to non-tumor nucleic acid from the subject; ranking the SVs wherein higher ranks are correlated to higher copy numbers; and providing reagents for an assay that detects a tumor signature comprising one or more of the SVs selected for having the higher ranks. Thus, methods may include determining copy number of detected SVs. Copy-number calling can then be performed to, for example, estimate tumor cell content in the sample and the degree to which the tumor genome may be rearranged. Genome-wide copy number information can be used later for prioritizing SVs for validation. Exemplary copy-number analysis can include ichorCNA described in Adalsteinsson, et al., 2017, Scalable whole-exome sequencing of cell-free DNA reveals high concordance with metastatic tumors, Nature Comm volume 8:1324, incorporated by reference.

Other criteria may also be used for selecting marker variants. For example, ranking may also include assigning a high rank to a truncal SV identified as an initiating truncal mutation of the tumor. The ranking step may include application of any other suitable criteria, such as the requirement for suitable primer binding sites by which one primer pair could amplify the multiple loci or instances of duplication of the marker variant. In certain tumor signature embodiments, the computer system is used to design multiple primer pairs that are useful to detect two or more of the SVs with higher ranks as a patient-specific, tumor-specific signature of the tumor in the subject.

### Assay Design

Methods may include designing and providing a plurality of copies of a primer pair that specifically amplify the sequence and storing the plurality of copies of a primer pair as reagents for use in one or more future assays for minimal residual disease. Designing the primer pair(s) may be implemented by a computer system. The computer system may also be used to design any or all other aspects of detection assay for the marker variant(s). The computer system may be used to design, for example, suitable fluorescent hydrolysis probes such as the probes sold under the trademark TAQMAN by Thermo Fisher Scientific (Waltham, MA). The computer system may be programmed to calculate and store conditions for a digital PCR (dPCR) assay such as: sample volume, dilution factor, partition number, reagent concentration, instrument settings, excitation and detection wavelengths, others, or any combination thereof. Output parameters from the computer system may be used as inputs to a suitable dPCR instrument or system, optionally carried from the assay design pipeline to the dPCR system by a direct data connection (e.g., WiFi or LAN) or by a managed system such as a laboratory information management system (LIMS).

From that, the computer system may thus provide conditions for an amplification reaction that will use a plurality of primer pairs designed to amplify a respective plurality of structural variants (SVs). The computer system output may also specific information such as sequences for the primer pairs that will amplify copies of the one or more SVs. Oligonucleotide reagents (primer pairs and fluorescent probes) maybe synthesized or obtained from a vendor such as Integrated DNA Technologies (Corralville, IA) and transferred (i.e., pipetted or dispensed) into reservoirs of the dPCR system. For example, the plurality of primer pairs are obtained as a reagent in one or more containers such as reagent tubes that are provisionally stored (e.g., lyophilized or in a freezer) and separately, subsequently dispensed to the detection assay instrument for use in the amplification reaction for detection of the plurality of SVs as a tumor-specific, patient specific signature of presences of the tumor.

### Detection Assay

Methods herein may include performing an assay with a high quantity of DNA as an input into the assay to detect a marker variant in a sample from a subject; and reporting the presence of the tumor in the subject when the assay is positive for the marker variant in the sample. The quantity of DNA used as input to the assay is preferably at least 100 ng, e.g., >500 ng including, e.g., 1 μg, and may include even up to 1.5 μg. That DNA used as input into the MRD detection assay may include cfDNA extract from a bodily fluid sample. The input DNA may include copies of cfDNA made by an amplification or pre-amplification. The input DNA may include both cfDNA extracted from a sample and copies made from among the cfDNA by a pre-amplification step. The pre-amplification step may be a linear amplification or PCR and may use primers specific to tumor sequences (e.g., ctDNA) and thus enrich for ctDNA among cfDNA.

The disclosed methods are useful for detection of any suitable target of interest in a sample. For example, methods are useful to detect nucleic acid from a pathogen in a mixed environmental sample or in a clinical sample that includes abundant host nucleic acid. The method may be used to detect fetal DNA in maternal blood or plasma. In certain preferred embodiments, the method is useful to detect a variant associated with a disease, such as an SV from tumor DNA present among cell-free DNA in a sample from a patient. Due to the high quantity of input DNA (e.g., up to 1.5 μg), the detection assay is suitable even where the ctDNA is vanishingly scarce. That includes conditions such as cfDNA leakage whereby a sample includes super-abundant cfDNA that is not ctDNA. That situation may occur after tissue trauma such as surgery, even surgery to remove a tumor. Especially in embodiments that further use a tumor-specific pre-amplification to enrich ctDNA relative to total cfDNA, methods of the invention provide very highly sensitive tests for MRD.

Any suitable sample may be used. For example, the sample may be blood, saliva, solid tissue, fine needle aspirate, a tumor biopsy (including material liberated from a formalin-fixed, paraffin embedded tumor sample), oral (e.g., buccal) swab, urine, stool, or any other sample. In certain embodiments, the sample comprises blood or plasma and the nucleic acid comprises circulating tumor DNA (ctDNA) among cell-free DNA (cfDNA) in the blood or plasma.

Methods of the invention may be used to detect any nucleic acid feature of interest including, for example, specific sequences, genes, or variants (e.g., mutations), which may include polymorphisms, small indels, or structural variants (which may include deletions, rearrangements, large indels, translocations, copy number variants, or others). In certain embodiments, the variant is a structural variant (SV) and a pre-amplification is performed with a PCR primer pair design to anneal to sites that flank a breakpoint of the SV. Preferred embodiments provide methods useful to detect nucleic acid fragments containing targeted structural variants present in a sample at low abundance (e.g., as low as one copy).

In preferred liquid biopsy and dPCR for MRD embodiments, the obtaining step may involve receiving one or more blood collection tubes or containers containing blood or plasma that was obtained from the subject via blood draw. The sample may include cell-free DNA from blood or plasma from the subject. The sample preferably includes at least about 100 ng of cfDNA (although in some embodiments, ctDNA is selectively enriched until at least about 100 ng of DNA is available). Whether or not a selected enrichment, e.g., pre-amplification, is preformed, methods including using at least about 100 ng as input, and preferably at least about 500 or more ng including, e.g., up to about 1.5 microgram of DNA, which is used as input to the MRD assay.

In certain optional in vitro “pre amplification” embodiment, the detection may proceed by at least two distinct stages or mechanisms that include (i) copying the marker variant using variant-specific primers and tailed primers to form tailed amplicons and (ii) amplifying the tailed amplicons in the presence of probes that indicate the presence of amplicons from the target of interest in the compartment. In some embodiments, a pre-amplification step may use primers designed to specifically amplify the marker variant. For example, if the variant is a structural variant, the pre-amplification may use a pair of primers designed to anneal to nucleic acid at locations that flank a breakpoint of the structural variant. This strategy further enriches the sample for copies of the marker variant, ensuring that the presence of the variant is detected in the subsequent detection steps. This pre-amplification step specifically addresses problems associated with some dead volume of sample that resists detection by existing digital PCR (dPCR) approaches. Due to the stochastic nature of sampling, some very minor fraction of a sample might otherwise, by chance, typically go undetected by dPCR. Here, the pre-amplification step may increase quantity of the marker variant prior to the partitioning and dPCR detection, reducing the likelihood that the target of interest will be undetected due to stochastic loss in dead volume. After the pre-amplification, the sample may be partitioned into compartments.

Regardless of any optional in vitro “pre-amplification” embodiments, the detection assay is useful to detect the tumor, and may be used as an MRD assay using at least 100 ng of DNA as input, preferably at least about 1 microgram and even, e.g., up to about 1.5 microgram as input. Targeting a variant (i.e. SV) that is unique to the tumor also allows high input where cfDNA is leaking. This means that saturation doesn't occur. If we targeted Wt and tumor DNA then partitions would saturate very easily even at low inputs. But because we are targeting only the tumor DNA then saturation doesn't occur because total tumor (ctDNA) is not high.

### High Sensitivity Mrd Assay

Methods may be used in assays for minimal residual disease (MRD), e.g., after a treatment to eradicate the tumor. Methods my include obtaining at least 100 ng cfDNA (optionally at least 500 or 1,000 ng) from a bodily fluid sample from a subject who has undergone treatment for a tumor; performing an amplification reaction in the sample using a primer pair that is designed to amplify a tumor-specific marker variant. Methods for detecting minimal residual disease (MRD) may include performing an MRD assay on a sample from a subject who has undergone cancer treatment to detect a set of selected marker variants that are in tumor nucleic acid and not in non-tumor nucleic acid of the subject; and reporting the presence of MRD in the subject when the assay is positive for at least one the marker variants in the sample, wherein the high quantity of input DNA maximizes sensitivity of the assay and/or makes the assay robust to cfDNA leakage.

Embodiments of the invention provide an MRD assay that uses up to 1.5 μg of cfDNA extracted from blood or plasma (or copies thereof) as a quantity of input DNA used in the assay. Preferably, sensitivity of the MRD is assay is less than about 10 ppm. Optionally, the invention provides an MRD assay with a sensitivity (LoD95) of <10PPM and a specificity of>99.5%. This is by performing the MRD result using structural variants and/or dPCR. Other factors include sequencing with coverage significantly less than 30× (preferably about 15×) to identify the variants for tracking. The disclosed assay may operate with a turn-around time from blood sample to result of <4 days. The assay may have a limit of blank of 0 and an MRD positive result may be reported for any signal above 0 (e.g., if any single compartment gives a positive result in digital PCR). The high sensitivity and robustness to cfDNA leakage is obtained by using a very high quantity of DNA as input into the MRD assay.

### Digital PCR

The described detection assay may be any suitable assay including, for example, nucleic acid sequencing, DNA microarray analysis, fluorescent in situ hybridization, PCR, quantitative PCR, or digital PCR (dPCR). In preferred embodiments, the detection assay is dPCR and the sample comprises blood or plasma from the subject and the assay uses at least 100 ng cfDNA (and/or copies made from cfDNA) as input. For the assay, dPCR may include partitioning at least a portion of the at least 100 ng into partitions that include PCR reagents and fluorescent probes for the amplicons and conducting the amplification reaction in the partitions. Where the input comprises 1.5 μg and steps are performed to protect against saturation, a portion of the 1.5 μg, e.g., 750 ng may be used as input into the dPCR. If the dPCR result is saturated (all or substantially all partitions blank), a second portion of the 1.5 μg may be taken, diluted, and partitioned for a second round of dPCR. The assay comprises performing an amplification reaction to detect amplification of the copies of the one or more SVs.

The assay includes partitioning the sample into solid, or optionally, partitions and performing an amplification reaction in the partitions using at least one primer specific for the sequence and a probe that provides a signal when the amplification reaction using at least one primer generates an amplification product. The method may include partitioning the sample into partitions that include PCR reagents and fluorescent probes for the amplicons, conducting the amplification reaction in the partitions, and detecting fluorescence from the partitions to detect the residual presence of the tumor after the treatment. Those dPCR steps may all be automated and/or performed using a commercially available dPCR instrument or system. The dPCR system may detect fluorescence from the partitions and provide output indicating a number of partitions that include the marker variant.

For a subject in whom a tumor has been diagnosed, and a sample of the tumor (biopsy, FFPE slice) obtained, a treatment may have been administered to eradicate the tumor. For example, the person may have undergone surgical resection to remove the tumor, radiation therapy to ablate the tumor, or chemotherapy to kill cells of the tumor. The person may spend some amount of time feeling the benefit of the treatment, living a cancer-free life. However, one issue with cancer treatment is that, after treatment to eradicate a tumor, non-tumor cells in the person may shed a super-abundant quantity of cell-free DNA that is not tumor DNA into circulation, a phenomenon known as cfDNA leakage. For the few days immediately after tumor resection/treatment, cfDNA leakage may mask the presence of ctDNA and inhibit the detection of MRD. In such situations, the invention provides methods that provide a detection assay that can detect MRD even in the presence of overwhelmingly abundant non-tumor cell-free DNA. Thus, an MRD assay of the invention may be beneficial in the first few hours to few days to ten days after treatment to remove a tumor, or when a person has suffered other, incidental tissue injury, among elderly people or a patient suffering arthritis or any other condition associated with inflammation. By using at least 100 ng and even up to 1.5 μg of DNA as input into the MRD assay, the assay design avoids false negatives, has very high sensitivity, and detects the presence even of tumor DNA even when present as only a very minor fraction of cfDNA.

Methods of the invention may be used to provide the elements of a detection assay (e.g., assay design and reagents such as PCR primers and detection probes) that may be kept and used repeatedly over time, e.g., tens of times or more, over months and years, conveniently and inexpensively. The detection assay may be a PCR-based assay that only needs a blood draw, as described, so that, after undergoing treatment, a person may know via a relatively quick, inexpensive, and minimally invasive test, whether there is any evidence of MRD.

The invention provides assays for minimal residual disease (MRD) that use at least 100 ng of cfDNA extracted from bodily fluid (preferably up to 1.5 μg) as input into digital PCR in which a sample is divided into compartments provided with PCR primers that are designed to specifically amplify circulating tumor DNA (ctDNA). PCR is performed with those primers and tumor-specific detectably-labeled probes such that detecting signal from the compartments provides evidence of ctDNA in the bodily fluid and thus evidence of MRD in the patient.

What is claimed is:

1. A method for detecting a tumor, the method comprising:
dividing at least a portion of a sample into partitions, wherein the sample comprises at least 100 ng of cell-free DNA from a subject;
conducting an amplification reaction in the partitions with reagents that specifically amplify tumor DNA and provide a detectable signal when the tumor DNA is amplified; and
reporting the presence of a tumor in the subject when the detectable signal is detected from the partitions.

2. The method of claim 1, further comprising performing a pre-amplification reaction in the portion of the sample using primers that specifically amplify the tumor DNA.

3. The method of claim 2, wherein after the pre-amplification, the portion of the sample includes at least 100 ng of nucleic acid comprising the cell-free DNA and product of the pre-amplification.

4. The method of claim 1, wherein after the pre-amplification, the portion of the sample includes at least 1 μg of nucleic acid that is divided into the partitions.

5. The method of claim 1, wherein the sample includes between and 1 and 2 μg of the cell-free DNA.

6. The method of claim 1, wherein the sample has been obtained within about 10 days or less after a treatment to remove a tumor from the subject.

7. The method of claim 1, wherein the method includes, when at least a majority of the partitions give the detectable signal in the subjecting step, taking a second portion of the sample, diluting the second portion, dividing the second portion in a second set of partitions and repeating the amplification reaction.

8. The method of claim 1, wherein the subject has undergone treatment to remove a tumor and the method further comprises—prior to the dividing step: analyzing tumor DNA from a tumor sample from the subject to detect tumor-specific sequences; designing primers specific for the tumor-specific sequences; and conducting the amplification reaction with the primers specific for the tumor-specific sequences.

9. The method of claim 8, wherein the analyzing step comprises:
sequencing tumor DNA from the tumor sample to obtain tumor sequence reads; and
comparing the tumor sequence reads to reference information to identify one or more variants in the tumor sequence reads, compared to the reference information.

10. The method of claim 9, further comprising filtering the identified one or more variants against data describing benign somatic variants to retain only tumor-specific variants as the tumor-specific sequences.

11. The method of claim 10, wherein the data describing the benign somatic variants comprise: matched-normal sequence reads from non-tumor tissue from the subject; and/or a database of somatic variants.

12. The method of claim 8, wherein the tumor-specific sequences comprise structural variants (SVs) specific to the tumor, or breakpoints of the SVs.

13. The method of claim 1, wherein the portion of the sample includes at least 1 μg of the cell-free DNA that is divided into the partitions.

14. The method of claim 1, wherein the dividing the portion of the sample into the partitions includes forming droplets or dividing the portion into wells of a plate, optionally wherein method includes analyzing the partitions with a digital PCR instrument.

15. The method of claim 1, wherein the partitions, the conditions for the amplification reaction, and the reagents that specifically amplify the tumor DNA constitute a digital PCR assay for minimal residual disease (MRD), and wherein the digital PCR assay for the MRD is performed using an input amount of DNA that is loaded into the partitions, wherein the input amount of the DNA is between about 1 and 2 μg, optionally between about 1 and 1.5 μg and optionally wherein the DNA comprises the cell-free DNA from a subject and/or product of a pre-amplification reaction that specifically pre-amplifies the tumor DNA from among the cell-free DNA.
